(19) United States
(12) Patent Application Publication
(10) Pub. No.: US 2010/0217309 A1
(43) Pub. Date: Aug. 26, 2010

(54) PLUG FOR ARTERIOTOMY CLOSURE AND METHOD OF USE

(75) Inventors: James G. Hansen, Coon Rapids, MN (US); Jason P. Hill, Brooklyn Park, MN (US); Christopher D. Johnson, Brooklyn Park, MN (US); Joel N. Groff, Montrose, MN (US)

Correspondence Address: CROMPTON, SEAGER & TUFTE, LLC, 1221 NICOLLET AVENUE, SUITE 800, MINNEAPOLIS, MN, 55403-2420, US

(73) Assignee: BOSTON SCIENTIFIC SCIMED, INC., Maple Grove, MN (US)

(21) Appl. No.: 12/389,960

(22) Filed: Feb. 20, 2009

(51) Int. Cl. A61B 17/03; B23P 17/04

(52) U.S. Cl. 606/213; 29/592

(57) ABSTRACT

The disclosure provides a modified arteriotomy closure plug, said modification selected from the group consisting of a slit, a notch, a groove, and combinations thereof and a method of manufacturing the arteriotomy closure plug.

### BACKGROUND OF THE INVENTION

A large number of diagnostic and interventional procedures involve the percutaneous introduction of instrumentation into a vein or artery. For example, coronary angioplasty, angiography, atherectomy, stenting of arteries, and many other procedures often involve accessing the vasculature through a catheter placed in the femoral artery or other blood vessel. Once the procedure is completed and the catheter or other instrumentation is removed, bleeding from the punctured artery must be controlled.

Traditionally, external pressure is applied to the skin entry site to stem bleeding from a puncture wound in a blood vessel. Pressure is continued until hemostasis has occurred at the puncture site. In some instances, pressure must be applied for up to an hour or more during which time the patient is uncomfortably immobilized. Further, external pressure to close the vascular puncture site works best when the vessel is close to the skin surface but may be unsuitable for patients with substantial amounts of subcutaneous adipose tissue since the skin surface may be a considerable distance from the vascular puncture site.

There are several approaches to close the vascular puncture site including the use of anchor and plug systems as well as suture systems. Internal suturing of the blood vessel puncture requires a specially designed suturing device. These suturing devices involve a significant number of steps to perform suturing and require substantial expertise. Additionally, when releasing hemostasis material at the puncture site and withdrawing other devices out of the tissue tract, the currently employed approaches to sealing the puncture may only partially occlude the tract thereby allowing blood to seep out of the puncture.

### SUMMARY

The disclosure relates to a plug for arteriotomy closure comprising an elongated member having a distal end, a proximal end, and an axial lumen connecting the distal end and the proximal end, said lumen sized to receive an axial suture, said elongated member including at least one modification selected from a slit, a notch, a groove, and combinations thereof.

In another aspect, the disclosure relates to a method of manufacturing a composite plug for arteriotomy closure comprising the steps of providing a blank larger than a desired plug; removing excess material from the blank larger than a desired plug to form a plug having a distal end and a proximal end; providing a lumen sized to receive a suture, said lumen connecting the distal end and the proximal end of the plug; providing at least one modification to the plug, said modification selected from a slit, a notch, a groove, and combinations thereof; providing a suture within the lumen which extends distally and proximately from the core member; and partially compacting the plug in a radial dimension.

In yet another aspect, the disclosure relates to a method of deploying a plug for arteriotomy closure comprising the steps of providing an elongate plug having a distal end, a proximal end, and a lumen connecting the distal end and the proximal end, said lumen sized to receive a suture wherein the plug includes a suture extending through the lumen; radially compacting the plug; inserting the compacted plug into a temporary containment sheath; providing an anchoring site adjacent to a vessel puncture; attaching the suture to the anchoring site; inserting the temporary containment sheath at least partially into a tract adjacent to the vessel; ejecting the plug from the temporary containment sheath; positioning the composite plug adjacent to the vessel and within the tract; tensioning the suture; axially compressing the plug, whereupon the plug buckles to at least partially fill the tract; and hydrating the plug.

### BRIEF DESCRIPTION OF DRAWINGS

FIG. 1 is a perspective view of an arteriotomy closure plug of the prior art.

FIG. 2 is a detail of a side view of a deployed arteriotomy closure plug of the prior art.

FIG. 3 is a side view of a comparative arteriotomy closure plug.

FIG. 4 is a side view of a comparative arteriotomy closure plug.

FIGS. 5A-E are sequential perspective views of an embodiment of the invention.

FIGS. 6A-E are sequential perspective views of an embodiment of the invention.

FIGS. 7A-E are sequential perspective views of an embodiment of the invention.

FIG. 8 is a schematic cross-section of a composite arteriotomy closure plug of the invention during delivery.

### DETAILED DESCRIPTION

The following description should be read with reference to the drawings wherein like reference numerals indicate like elements throughout the several views. The drawings, which are not necessarily to scale, are not intended to limit the scope of the claimed invention. The detailed description and drawings illustrate example embodiments of the claimed invention.

All numbers are herein assumed to be modified by the term “about.” The recitation of numerical ranges by endpoints includes all numbers subsumed within that range (e.g., 1 to 5 includes 1, 1.5, 2, 2.75, 3, 3.80, 4, and 5).

As used in this specification and the appended claims, the singular forms “a”, an and “the” include the plural referents unless the content clearly dictates otherwise. As used in this specification and the appended claims, the term “or” is generally employed in its sense including “and/or” unless the content clearly dictates otherwise.

Embodiments are described herein in the context of a hemostatic pressure plug. Those of ordinary skill in the art will appreciate that the following detailed description is illustrative only and is not intended to be in any way limiting. In the interest of clarity, not all of the routine features of the implementations described herein are shown and described.

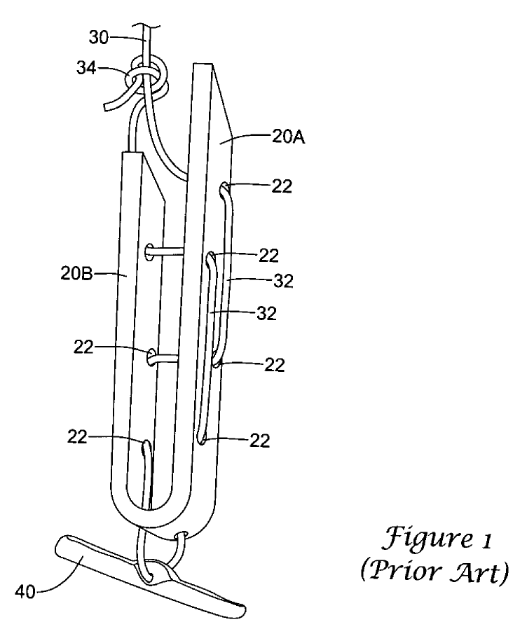

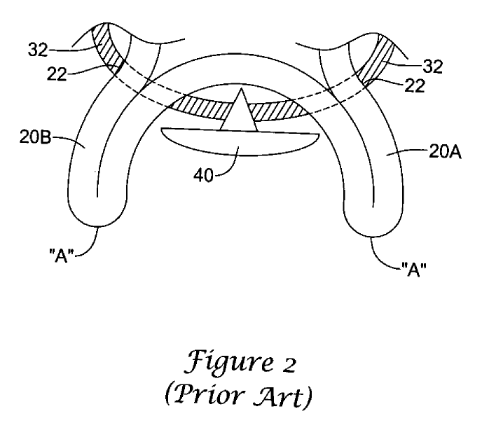

Referring now to FIGS. 1 and 2, which together illustrate an arteriotomy closure plug of the prior art, suture (30) passes alternately through holes (22) to form stitches (32) which secure folded collagen strip (20A, 20B) and anchoring toggle (40) before terminating in a slip knot (34). FIG. 2 illustrates a distal detail of the arteriotomy closure plug in a deployed condition in which stitches (32) have been overtightened resulting in protruding regions “A” which may undesirably enter the vessel.

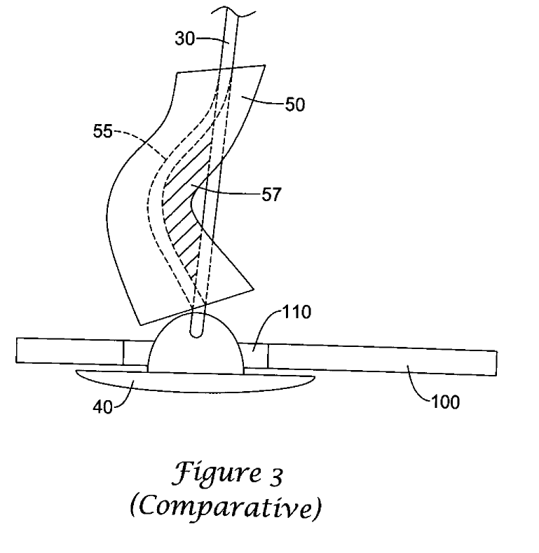

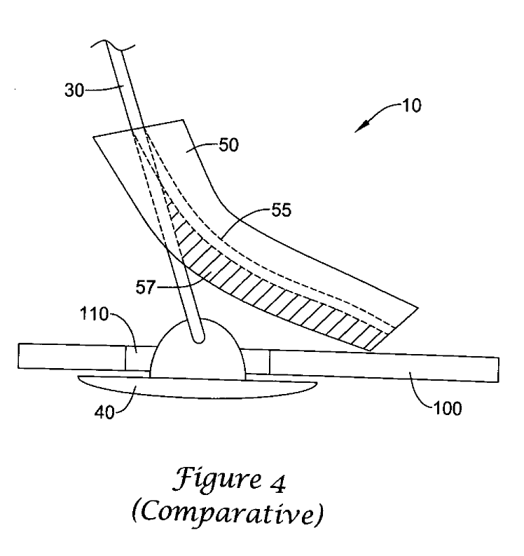

Comparative FIGS. 3 and 4 illustrate possible failure modes which may be encountered when using unmodified closure plugs similar to those of the invention. Plugs (50), having an axial lumen (55) configured to accept suture (30) have been positioned adjacent to opening (110) in vessel wall (100). Suture (30) has been attached to anchor (40) and lightly tensioned. Compression of unmodified plugs (50) within the wound tract (not shown) has resulted in tearing (57) of the plugs (50) and improper positioning of the plug material as well as the possible generation of debris within the wound.

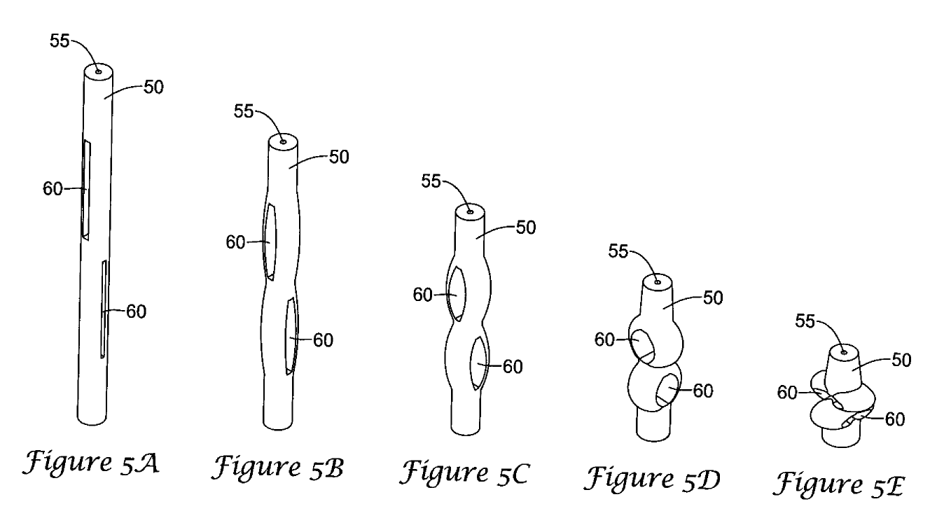

FIGS. 5A-E illustrate progressive stages in the axial compression of a modified a arteriotomy closure plug of the invention in which the modification is in the form of vertical slits (60) which allow the plug (50) to buckle in a controlled manner thereby filling the wound tract while reducing the pressure applied to the region near the vessel opening and avoiding or minimizing tearing of the suture lumen (55).

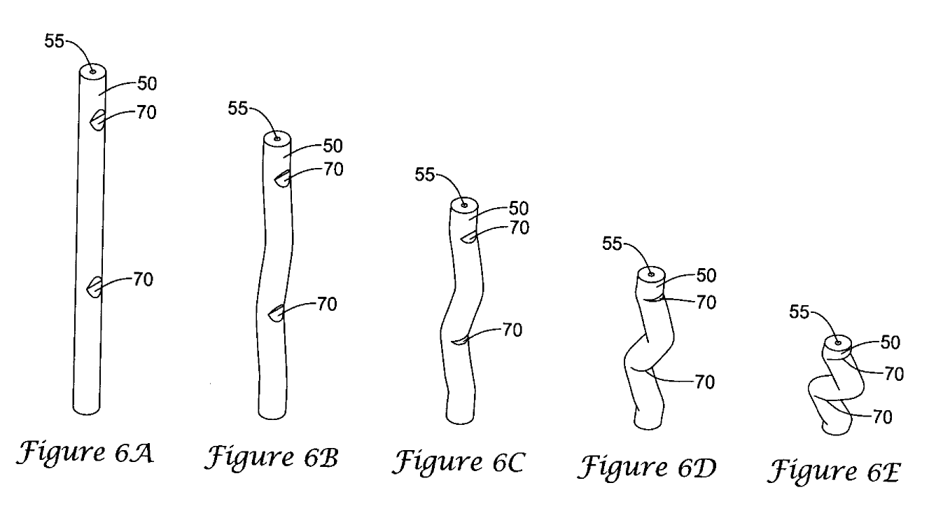

FIGS. 6A-E illustrate progressive stages in the axial compression of one embodiment of the invention in which the modification is in the form of V-notches (70) which allow the plug (50) to buckle in a controlled manner thereby filling the wound tract while reducing the pressure applied to the region near the vessel opening and avoiding or minimizing tearing of the suture lumen (55).

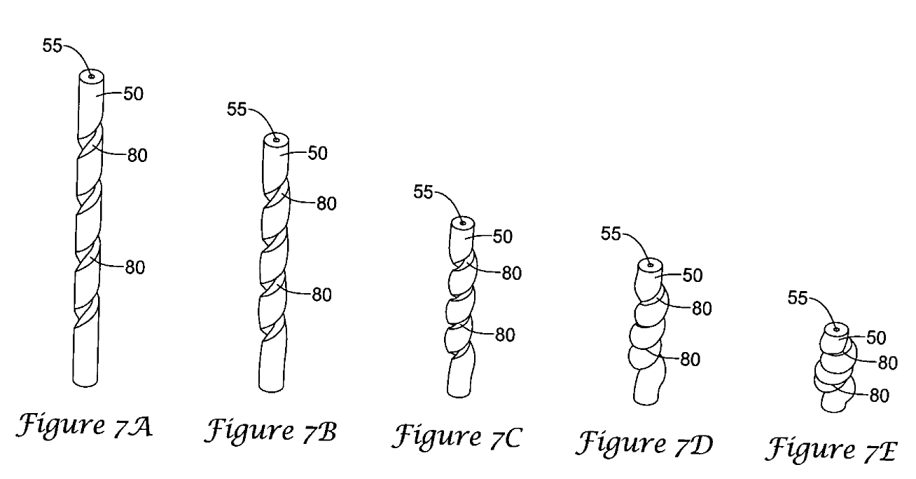

FIGS. 7A-E illustrate progressive stages in the axial compression of one embodiment of the invention in which the modification is in the form of spiral V-groove (80) which allow the plug (50) to buckle in a controlled manner thereby filling the wound tract while reducing the pressure applied to the region near the vessel opening and avoiding or minimizing tearing of the suture lumen (55).

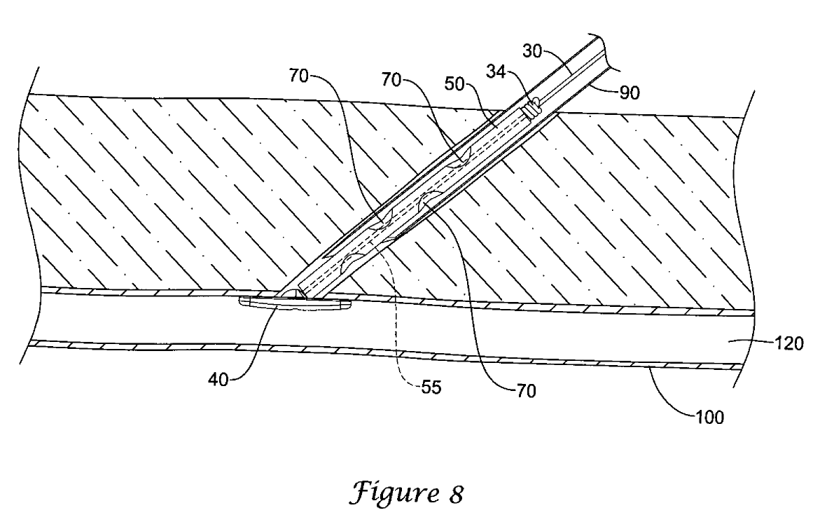

FIG. 8 illustrates schematically the deployment of a modified plug (50) for arteriotomy closure within a wound tract adjacent to an arterial puncture. The arterial wall (100) has been punctured proximate the anchoring site. In this illustrative example, suture (30) has been attached to a toggle (40) or patch disposed within the lumen (120) of the artery. The plug (50) has been modified by the addition of notches (70) and radially compressed within temporary sheath (90) and advanced through the tract to a position adjacent to the arterial puncture. The modified plug (50) may then be ejected from the temporary sheath (90) by conventional means, which have been omitted for clarity, and the temporary sheath may be withdrawn, typically through a cannula within the wound tract. Following or concurrent with ejection, the composite plug may be axially compressed within the wound tract and allowed to buckle within the wound tract. A variety of techniques may be used alone or in combination to effect compression and buckling. For example, the axial suture (30) may be placed in tension against toggle (40) while a slip knot (34) is advanced distally along the suture. Suitable slip knot configurations are described in co-pending application Ser. No. ______, filed ______ incorporated herein by reference in its entirety. Compression within the tract may be augmented by the use of a pushrod. The presence of slits, notches, grooves, helical grooves, and the like may facilitate axial compression and folding in a controlled manner while minimizing forces which might tend to result in undesirable intrusion of the plug into the vessel.

As noted above, in some embodiments one or more regions of the plug may be modified by providing slits or material may be removed to provide notches, grooves, helical grooves, and the like which may lower axial compression forces on the composite plug during deployment within a wound tract by allowing the plug to buckle. Smooth compression is desirable to control buckling and folding of the composite plug while avoiding excessive internal stresses which may tend to allow the suture to tear the composite plug. A plug which is asymmetrically notched or grooved may be viewed as an eccentrically loaded column. Similarly, slits may be viewed as creating adjacent columns of reduced effective diameter (greater slenderness) and may also create an eccentric loading pattern. A column modified at multiple sites by slits, notches or grooves often will buckle in a manner which reflects an initial buckling at one modification site followed quickly by further buckling at additional modification sites as the initially asymmetric loading is accentuated by buckling at adjacent modification sites. In any event, the column may be viewed as having one or more significantly reduced critical buckling loads. In addition to facilitating axial compression, the removed regions may provide improved fluid access to the interior of the plug including, but not limited to, access to any interior layers which may be present therein. Such fluid access may be used to accelerate local swelling.

It is believed that the ability of an arteriotomy closure plug to seal a wound tract may have several major contributors. The first is simply the fraction of the cross-section of the tract which is occluded by the plug. Early axial buckling of the plug upon deployment effectively increases the fraction of the wound tract which is occupied by the plug. Selection of an appropriate material as the major component of the plug will further increase the occlusion of the tract as the selected material swells upon exposure to bodily fluids or externally introduced water. For example, commercially available collagen strips may exhibit free volumetric swelling ratios as low as about 3:1 to about 5:1 while properly prepared gelatin foam plugs may exhibit swelling ratios of about 14:1, about 18:1 or even 100:1. Other materials which may be present in or on the plug also may contribute to wound sealing by swelling, by promoting clotting, and/or by increasing the viscosity of fluids within the tract.

In some embodiments, the modification to the plug is in the form of a notch or groove having a generally U-shaped or V-shaped cross-section although other notch or groove shapes are within the scope of this disclosure. The notch or groove may be generally in a plane which is perpendicular to the axis of the plug or at an angle to the axial direction. In those embodiments in which the modification includes multiple notches or grooves, including those embodiments in which the groove is in the form of a helix, the spacing of the notches or grooves may be uniform or nonuniform. Additionally, the distribution of notches or grooves may be circumferentially uniform or may be asymmetric. In some embodiments, a notch or groove may intersect another notch or groove. The pattern of notches or grooves may produce a generally planar zigzag folding pattern or may produce a generally corkscrew-like column upon buckling. In some embodiments, the buckling pattern may result in an axially projected cross-section which is generally circular about the suture line while in other embodiments the axially projected cross-section may include lobes. Similarly, when the modification is in the form of a slit or slits, the slit may be oriented axially, transversely, or at an intermediate angle. The plane of the slit may intersect the axis of the plug or not as desired. As in the case of notches or grooves, slits may be distributed uniformly or nonuniformly and may intersect if desired.

In many embodiments, the plug of the disclosure is configured to buckle in the vicinity of the modifying feature and to assume a predetermined configuration when the plug buckles upon axially compression. In general, buckling of plugs which have been modified by the inclusion of a slit, notch or groove and combinations thereof requires less axial force than would be required to buckle an unmodified plug of similar construction and that the reduction of axial force lessens the risk of accidental protrusion of the plug into the vessel adjacent to which it is being deployed. Periodic buckling of the plug may reduce the maximum lateral excursions experienced by portions of the plug allowing the suture to remain generally aligned along the suture within the wound tract while minimizing or eliminating tearing in the vicinity of the suture lumen. In addition to the introduction of buckling modifications, it may be useful to modify the material of the closure plug as described in co-pending application Ser. No. ______, filed ______ incorporated herein by reference in its entirety.

In some embodiments, the arteriotomy closure plug may be a composite plug comprising one or more core members and one or more outer members. In addition, the plug may include one or more layers including a hydrogel, a hemostatic material, or a combination thereof. Plugs suitable for modification in the manner of this disclosure may be found, for example, in co-pending application Ser. No. ______, filed ______ incorporated herein by reference in its entirety.

Once fabricated and compressed, the arteriotomy closure plug may be inserted into a temporary containment sheath for delivery to the vicinity of a vessel puncture. The distal end of the suture may be attached to an anchor adjacent to the vessel puncture. The anchor site may be any of the anchor sites known in the art. For example, it may take the form of a loop attached to a toggle or patch located within the punctured vessel. Following insertion of the temporary containment sheath within the wound tract and tensioning of the distal suture to ensure that the composite plug is properly positioned within the tract, the plug may be ejected from the temporary containment sheath and, if necessary, further oriented within the tract by manipulation of the proximal end of the plug as it begins to expand. In addition, the plug may be axially compressed with attendant buckling and or folding within the wound tract. The containment sheath, as well as any other apparatus remaining within the wound tract, may then be removed to allow the composite plug to expand and seal the tract as it hydrates.

Various modifications and alterations of this invention will become apparent to those skilled in the art without departing from the scope and principles of this invention, and it should be understood that this invention is not to be unduly limited to the illustrative embodiments set forth hereinabove. All publications and patents are herein incorporated by reference to the same extent as if each individual publication or patent was specifically and individually indicated to be incorporated by reference.

1. A plug for arteriotomy closure comprising:
an elongated member having a distal end, a proximal end, and an axial lumen connecting the distal end and the proximal end, said lumen sized to receive an axial suture, said elongated member including at least one modification selected from the group consisting of a slit, a notch, a groove, and combinations thereof.

2. The plug of claim 1, wherein the modification is a notch having a generally U-shaped cross-section.

3. The plug of claim 1, wherein the modification is a notch having a generally V-shaped cross-section.

4. The plug of claim 1, wherein the modification is a groove having a generally U-shaped cross-section.

5. The plug of claim 1, wherein the modification is a groove having a generally V-shaped cross-section.

6. The plug of claim 1, wherein the modification is a helical groove having a generally U-shaped cross-section.

7. The plug of claim 1, wherein the modification is a helical groove having a generally V-shaped cross-section.

8. The plug of claim 1, wherein the modification is a star comprising crossed notches.

9. The plug of claim 1, wherein the elongated member comprises a core member and an outer member.

10. The plug of claim 1, wherein the elongated member comprises gelatin foam.

11. The plug of claim 10, wherein at least one of a hydrogel and a hemostatic material is distributed throughout a portion of the plug.

12. The plug of claim 1, wherein the elongated member comprises one or more layers including a hydrogel, a hemostatic material, and combinations thereof.

13. The plug of claim 12, wherein the one or more layers are external to the plug.

14. The plug of claim 12, wherein at least a portion of the one or more layers is disposed within the plug.

15. The plug of claim 1, wherein the plug assumes a predetermined configuration when the plug buckles upon axially compression.

16. The plug of claim 15, wherein the plug is configured to buckle in the vicinity of a modification selected from the group consisting of a slit, a notch, a groove, and combinations thereof when subjected to axial compression.

17. The plug of claim 15, wherein the axial force required to buckle the plug including at least one modification selected from the group consisting of a slit, a notch, a groove, and combinations thereof is less than the axial force required to buckle an unmodified plug of similar construction.

18. The plug of claim 15, wherein the axial suture remains generally straight as the plug assumes a predetermined configuration when the plug is axially compressed.

19. A method of manufacturing a composite plug for arteriotomy closure comprising the steps of:
providing a blank larger than a desired plug;
removing excess material from the blank larger than a desired plug to form a plug having a distal end and a proximal end;
providing a lumen sized to receive a suture, said lumen connecting the distal end and the proximal end of the plug;
providing at least one modification to the plug, said modification selected from the group consisting of a slit, a notch, a groove, and combinations thereof;
providing a suture within the lumen which extends distally and proximately from the core member; and
partially compacting the plug in a radial dimension.

20. The method of claim 19, wherein the blank is a gelatin foam.

21. The method of claim 19, further comprising the steps of:
providing a second blank;
removing excess material from the blank to form a second component of the plug;
providing a second suture lumen within the second component;
joining the plug and the second component to form a composite plug having a suture lumen extending from the distal end to the proximal end of the composite plug;
prior to the steps of providing at least one modification to the plug, said modification selected from the group consisting of a slit, a notch, a groove, and combinations thereof;
providing a suture within the lumen which extends distally and proximately from the core member; and
partially compacting the plug in a radial dimension.

22. The method of claim 19, further comprising the step of mechanically modifying at least a portion of the plug to alter the permeability of the plug to fluids.
